(19) United States
(12) Patent Application Publication
(10) Pub. No.: US 2020/0210917 A1
(43) Pub. Date: Jul. 2, 2020

(54) APPARATUS, METHOD, PROGRAM, SIGNAL FOR DETERMINING INTERVENTION EFFECTIVENESS INDEX

(71) Applicant: OMRON Corporation, KYOTO (JP)

(72) Inventors: Yasuyo KOTAKE, Shinagawa-ku, Tokyo (JP); Hiroshi NAKAJIMA, Kyoto-city, KYOTO (JP); Danni WANG, Nara-shi, NARA (JP)

(73) Assignee: OMRON Corporation, KYOTO (JP)

(21) Appl. No.: 16/639,133

(22) PCT Filed: Sep. 1, 2017
(86) PCT No.: PCT/IB2017/055270

§ 371 Date: Feb. 14, 2020

(51) Int. Cl. G06Q 10/06; A61B 5/16; A61B 5/18

(52) U.S. Cl. CPC G06Q 10/0639; A61B 2503/20; A61B 5/18; A61B 5/165

(57) ABSTRACT

A method for determining an intervention effect index for a person executing a task is provided and comprises: obtaining sensing information by at least one sensor coupled to the person, the sensing information includes first sensing information relating to performance in executing the task and second sensing information relating to an emotional state; determining, on the basis of said first sensing information, a performance value difference indicating a variation between performance in executing the task before and after an intervention is applied, the intervention representing an excitation affecting the person; estimating, on the basis of said second sensing information, an emotional value difference indicative of a variation between emotional states before and after the intervention is applied; determining the intervention effect index on the basis of said performance value difference and said emotional value difference, the intervention effect index representing an indication on effectiveness of the intervention on the person.

### TECHNICAL FIELD OF THE INVENTION

The present invention relates to an apparatus, a method, a program, and a signal for determining an intervention effectiveness index for a person executing a task.

### BACKGROUND ART

Methods for improving performance in systems comprising several device components, like a production line, are known in the art. For example, Japanese Patent No. 5530019 discloses a solution for detecting a sign of equipment malfunction, which is important to prevent the operational efficiency from decreasing, e.g. to prevent decrease in factory productivity. Further, in a production line involving an operation performed by a worker, factors known to influence the productivity, or specifically the quality and the amount of production, include 4M (machines, methods, materials, and men) factors. Three of these factors, namely, machines, methods, and materials (3M), have been constantly improved and enhanced to increase the productivity. However, the factor “men” depends on several elements, and has been more difficult to objectively address. For instance, in the field of controlling a manufacturing line, typically, a manager visually observes the physical and mental states of the worker, and provides an appropriate instruction for the worker to maintain and enhance the productivity. Such supervision is however prone to errors, and often based on a subjective contribution by the manager. As a result, it is difficult to objectively and systematically implement solutions aimed at improving productivity of the “men” factor, and in general it is difficult to further increase productivity/efficiency in systems wherein a person interacts with one or more machines in order to accomplish a task.

### BACKGROUND TO THE INVENTION AND TECHNICAL PROBLEM

In order to improve efficiency of a system like a production line by addressing the “men” factor, it is conceivable providing a worker with an intervention (e.g. an excitation signal), wherein the intervention is determined on the basis of the worker's mental state obtained from measurements, see for instance Japanese patent application JP2017037287 filed on 28 Feb. 2017, as well as PCT application having reference/docket number 198 761 and filed by the same applicant as the present application. More in particular, in order to accurately estimate the mental state of the person, it is advantageous estimating the emotional state on the basis of specific measurements, see e.g. Japanese patent application JP2017037306 filed on 28 Feb. 2017 and PCT application by same applicant filed on the same applicant as the present application and having reference/docket number 198 760, as well as above referred JP2017037287 and respective PCT (CASE #3 again). A suitable model for the emotional state and for its measurement-based estimation is also later illustrated. Thanks to the use of measurements, the emotional state can be accurately estimated in an objective and repeatable manner, such that it is possible to objectively and systematically provide an intervention leading to increased efficiency.

By means of the above, it is thereby possible to increase the efficiency of a system with which the person interacts.

However, while a general efficiency improvement is achieved by the above mentioned technique, it remains difficult to objectively determine the degree of such improvement, e.g. whether the improvement is very high or moderate; in other words, even if an improvement is achieved when using an intervention, the inventors have recognized that there is margin for a further efficiency increase, since it is possible at least in certain circumstances to apply an intervention leading to even higher efficiency than another intervention.

It is thus desirable to estimate how effective an intervention is, such that for instance the efficiency gain can be predicted, or such that for instance a specific intervention can be chosen instead of another intervention. One conceivable way of determining the intervention effectiveness lies in relying on a trained observer, who bases his/her guess on his/her experience and on observations made on the worker after the intervention is applied. This is however prone to errors, like in prior art techniques relying on a line manager or supervisor, and also entails a subjective component. Significantly, such a solution would not be suitable for implementation in a technical system, requiring an objective and repeatable way for autonomously determining the degree of effectiveness of an intervention.

Thus, existing solutions do not allow for a further improvement of efficiency. One problem also lies in how to objectively determine effectiveness of an intervention when wanting to further increase a system efficiency.

Thus, existing solutions do not allow further increasing efficiency in systems wherein a person interacts with one or more machines in order to accomplish a task, and do not allow for an accurate and objective determination of the degree of effectiveness in increasing efficiency.

### SUMMARY OF THE INVENTION

One of the objects of the present invention is thus to overcome the problems of the prior art.

Further aspects are herein described, numbered as A1, A2, etc. for convenience:

According to aspect A1, it is provided a method for determining an intervention effect index for a person executing a task, the method comprising steps of:
obtaining sensing information by means of at least one sensor coupled to the person, wherein the sensing information includes first sensing information relating to performance in executing the task and second sensing information relating to an emotional state;
determining, on the basis of said first sensing information, a performance value difference indicating a variation between performance in executing the task before an intervention is applied and performance in executing the task after the intervention is applied, the intervention representing an excitation affecting the person;
estimating, on the basis of said second sensing information, an emotional value difference indicative of a variation between emotional states before and after the intervention is applied;
determining the intervention effect index on the basis of said performance value difference and said emotional value difference, the intervention effect index representing an indication on effectiveness of the intervention on the person.

A2. The method according to aspect A1, further comprising the step of:
storing, in a database, at least one of a plurality of intervention information with a respective intervention effect index determined from said determining step for determining the intervention effect index.

A3. The method according to aspect A1 or A2, further comprising selecting, from a database, an intervention based on a predetermined intervention effect index.

A4. The method according to aspect A3, further including a step of applying the selected intervention.

A5. The method according to any of the preceding aspects, wherein the task includes a manufacturing task within a manufacturing line, and the intervention includes at least one amongst an intervention provided to the person and an intervention provided to at least one component included in the manufacturing line.

A6. The method according to any of the preceding aspects, wherein the task includes an operation for driving a vehicle, and the intervention includes at least one amongst an intervention directly provided to the person and an intervention provided to at least one component included in the vehicle.

A7. The method according to any of the preceding aspects, wherein the task includes an activity performed by the person, and the intervention includes a healthcare support feedback provided to the person.

A8. The method according to any of the preceding aspects, further including a step of monitoring effectiveness of an intervention on the basis of the determined intervention effect index.

A9. The method according to any of the preceding aspects, wherein the step of determining the performance value difference comprises:
determining a first performance value in executing the task based on said first sensing information obtained before the intervention is applied,
determining a second performance value in executing the task based on said first sensing information obtained after the intervention is applied,
determining said performance value difference (ΔM) based on the difference between said first performance value and said second performance value.

A10. The method according to aspect A2, further comprising the steps of:
determining an intervention effect index value in correspondence of a person;
selecting, from the database, an intervention to be applied to a person based on the determined intervention effect index value.

A11. The method of aspect 2, wherein the database further stores attributes of a person in association with said intervention and said respective intervention effect index, the method comprising selecting an intervention on the basis of at least one of said stored attributes.

A12. An apparatus for determining an intervention effect index for a person executing a task, the apparatus comprising:
an interface configured to obtain sensing information by means of at least one sensor coupled to the person, wherein the sensing information includes first sensing information relating to performance in executing the task and second sensing information relating to an emotional state;
a first processor configured to determine, on the basis of said first sensing information, a performance value difference indicating a variation between performance in executing the task before an intervention is applied and performance in executing the task after the intervention is applied, the intervention representing an excitation affecting the person;
a second processor configured to estimate, on the basis of said second sensing information, an emotional value difference indicative of a variation between emotional states before and after the intervention is applied;
a third processor configured to determine the intervention effect index on the basis of said performance value difference and said emotional value difference, the intervention effect index representing an indication on effectiveness of the intervention on the person.

A13. The apparatus according to aspect A12, further comprising a storage unit configured to store at least one of a plurality of intervention information with a respective intervention effect index determined by the third processor.

A14. The apparatus according to aspect A12 or A13, further comprising a selector configured to select, from a database, an intervention based on a predetermined intervention effect index.

A15. The apparatus according to aspect A14, further comprising an intervention applicator configured to apply the selected intervention.

A16. The apparatus according to any of aspect A12 to A15, wherein the task includes a manufacturing task within a manufacturing line, and the intervention includes at least one amongst an intervention provided to the person and an intervention provided to at least one component included in the manufacturing line.

A17. The apparatus according to any of aspects A12 to A16, wherein the task includes an operation for driving a vehicle, and the intervention includes at least one amongst an intervention directly provided to the person and an intervention provided to at least one component included in the vehicle.

A18. The apparatus according to any of aspects A12 to A17, wherein the task includes an activity performed by the person, and the intervention includes a healthcare support feedback provided to the person.

A19. The apparatus according to any of aspects A12 to A18, further including a monitoring unit configured to monitor effectiveness of an intervention on the basis of the determined intervention effect index.

A20. The apparatus according to any of aspects A12 to A19, wherein the first processor is further configured to:
determine a first performance value in executing the task based on said first sensing information obtained before the intervention is applied,
determine a second performance value in executing the task based on said first sensing information obtained after the intervention is applied,
determine said performance value difference based on the difference between said first performance value and said second performance value.

A21. The apparatus according to aspect A13, further comprising a processor configure to:
determine an intervention effect index value in correspondence of a person;
select, from the database, an intervention to be applied to a person based on the determined intervention effect index value.

A22. The apparatus of aspect A13, wherein the database further stores attributes of a person in association with said intervention and said respective intervention effect index, the apparatus is configured to select an intervention on the basis of at least one of said stored attributes.

A23. A non-transitory computer readable medium stored with a computer program including instructions which, when executed on a computer, cause the computer to execute the steps according to any of aspects A1 to A11.

A24. A signal carrying instructions which, when executed on a computer, cause the computer to execute the steps according to any of aspects A1 to A11.

A25. A system including an apparatus according to any of aspects A12 to A22, and a portable intervention applicator configured for being coupled to a person, wherein the portable intervention applicator is configured to apply to the person an intervention on the basis of information received from the apparatus.

### LIST OF FIGURES

FIG. 1 illustrates a block diagram of a mental state model that is well suited for technical applications wherein a person interacts with a device/machine.

FIG. 2 shows how cognitive and emotional states can be measured by way of objective and repeatable measurements.

FIG. 3 shows examples of objective and repeatable measurements.

FIG. 4A is a flow chart illustrating a method according to a first embodiment of the present invention.

FIG. 4B is a flow chart illustrating optional variants of the method according to a first embodiment of the present invention.

FIG. 5 is a diagram showing an image captured when a person has completed a task, the image showing the outcome or result of the completed task.

FIG. 6 is a schematic diagram of a manufacturing line, representing an example of a system including one or more machines with which a person can interact.

FIG. 7A is a block diagram illustrating an apparatus (entity) according to a second embodiment of the present invention.

FIG. 7B is a block diagram illustrating an apparatus (entity) according to an optional variant of a second embodiment of the present invention.

FIG. 8 is a block diagram illustrating a computer suitable for carrying out the invention according to an embodiment of the present invention.

FIG. 9 is a block diagram illustrating a system according to an embodiment of the present invention.

FIG. 10 is a block diagram illustrating how an emotional state can be objectively estimated on the basis of measurement.

FIGS. 11A and 11B are examples provided to show how performance and emotional state may vary over time and in correspondence of an applied invention.

FIGS. 12A and 12B are further examples provided to show how performance and emotional states vary over time, and how the measured and estimated values can be used to calculate the WI index.

FIG. 13 is an example showing how effectiveness varies over time.

### DETAILED DESCRIPTION

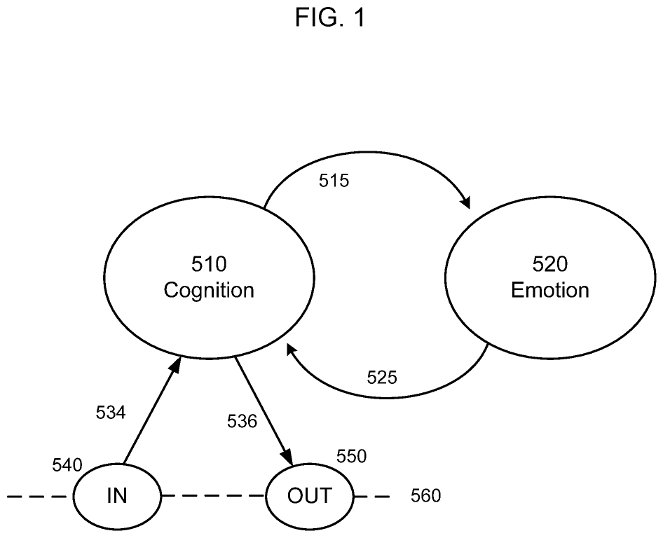

Before entering into the details of the embodiments, reference will be made to FIGS. 1 to 3 illustrating how a mental state can be described conveniently by a model, which has been recognized by the inventors as being suitable for handling a mental state of a person in an objective and repeatable manner that can be conveniently used in an autonomous computing apparatus or entity.

Human performance can be defined as the efficiency and/or effectiveness exhibited by a person when performing a certain activity or task. Efficiency can be considered as the time required for completing the activity, or similarly the speed in completing the activity. Effectiveness indicates how well the activity is completed, e.g. how close the outcome of the activity is to an expected outcome of the same activity. Let us consider the case wherein the person is, for example, an operator on a production line, and the activity is one task required to be performed along the production line to obtain a produced item. In this example, the time required to complete a certain task along a production line represents an example of efficiency; the yield achieved when performing such a task represents instead an example of effectiveness, wherein the yield indicates for instance a percentage of items produced that satisfy certain predetermined parameters over the entire number of produced items (in a given time unit; and/or in cumulated total for the person, a group of persons, etc.).

In the prior art, there is a limit to improving the performance at the production site. In fact, the performance of an operator on such a production line has been increased in the art by means of techniques like improving the layout of the line, providing improved tools, providing training so that the operator's ability increases. However, known methods reach their limits, such that a further improvement is difficult to achieve.

The present invention is based, amongst others, on the recognition that human state can be described by an appropriate model that takes into account different types of states of a person, wherein the states are directly or indirectly measurable by appropriate sensors. Also, the human state plays an important role in the efficiency and effectiveness of the person executing a task. Thus, human performance can be objectively and systematically observed, and appropriately improved according to the inventors.

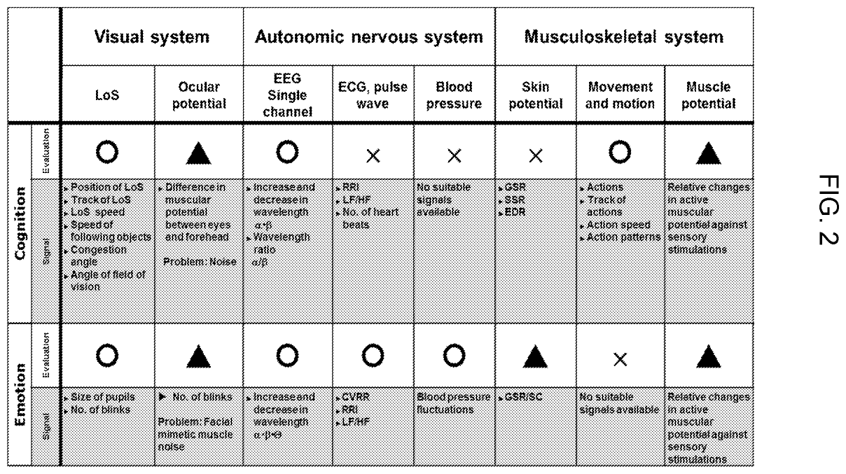

More in detail, it has been recently shown that human state depends on aspects like cognitive state and emotional state of a person. While for the later discussion emotional states are more relevant, both cognitive and emotional states are herein discussed for the sake of completeness. The cognitive state of the person relates to, for example, a state indicating a level of ability acquired by a person in performing a certain activity for instance on the basis of experience (e.g. by practice) and knowledge (e.g. by training). The cognitive state is directly measurable, since it directly relates to the execution of a task by the person. Emotional state has been considered in the past solely as a subjective and psychological state, which thus could be assessed only subjectively by the person. Other (more recent) studies however led to a revision of such old view, and show in fact that the emotional state of a person are presumed to be hard wired and physiologically (i.e. not culturally) distinctive; further, being based also on arousal (i.e. a reaction to a stimuli), emotions can be indirectly obtained from measurements of physiological parameters objectively obtained by means of suitable sensors, as also later mentioned with reference to FIG. 2.

The human model as herein adopted consists of emotion and cognition, and cognition functions as an interface with the outside world by input (stimulation) and output (physiological parameter). Emotion and cognition are affected by input; output is the result of interaction between emotion and cognition, wherein both can be measured. More in detail, FIG. 1 shows a model that can be used, according to the inventors, for technically assessing human performance. In particular, the model comprises a cognitive part and an emotional part interacting with each other. The cognitive part and the emotional part represent the set of cognitive states and, respectively, the set of emotional states that a person can have, and/or that can be represented by the model. The cognitive part directly interfaces with the outside world, in what the model represents as input and output. The input represents any stimuli that can be provided to the person, and the output represents any physiological parameters produced by the person, and as such measurable. The emotional part can be indirectly measured, since the output depends on a specific emotional state at least indirectly via the cognitive state, according to the model of FIG. 1. In other words, an emotional state will be measurable as an output, even if not directly due to the interaction with the cognitive part. It is herein not relevant how the cognitive part and the emotional part interact with each other, and it is in fact left to theories and studies that are not part of the invention. What matters to the present discussion is that there are input to the person (e.g. stimuli or excitations) and output from the person as a result of a combination of a cognitive state and an emotional state, regardless of how these interact with each other. In other words, the model can be seen as a black box having objectify measurable input and output, wherein the input and output are causally related to the cognitive and emotional states, though the internal mechanism for such causal relationship are herein not relevant.

Despite the non-knowledge of the internal mechanisms of the model, the inventors have noted that such a model can be useful in practical application in the industry, like for instance when wanting to increase efficiency on a production line, as it will also become apparent in the following.

It is herein noted that various sensors can be used for cognitive and emotional measurement. More in detail, FIG. 2 shows how cognitive and emotional states can be measured by way of objective and repeatable measurements, wherein a circle, triangle and cross indicates that the listed measuring methods are respectively well suitable, less suitable (due for instance to inaccuracies), or (at present) considered not suitable. Other techniques are also available, like for instance image recognition for recognizing facial expressions or patterns of facial expressions that are associated to a certain emotional state. In general, cognitive and emotional states can be measured by an appropriate method, wherein certain variable(s) deemed suitable for measuring the given state are determined, and then measured according to a given method by means of suitable sensor(s). A variety of sensors are suitable for obtaining such measurements, and are herein not described since any of them is suitable as long as they provide any of the parameters listed in FIG. 2, or any other parameters suitable for estimating cognitive and/or emotional states. The sensors can be wearables, e.g. included in a wrist or chest wearable device or in glasses, an helmet like device for measuring brain activity from the scalp (e.g. EEG/NIRS), or a large machine like PET/fMRI.

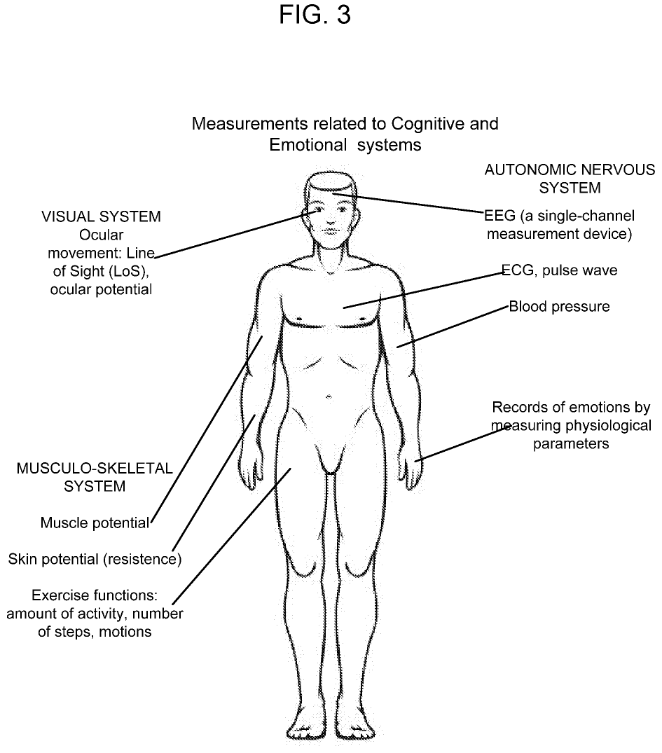

Thus, it possible to model a person, like for instance an operator of a factory production line, by using a model as illustrated in FIG. 1, and by collecting measurements of physiological parameters of the person as shown in FIGS. 2 and 3.

As also later detailed, in particular, it is possible to estimate the emotional state of a person in an objective and autonomous manner.

Starting from the above considerations, it is thus proposed a method, apparatus (entity), computer program and system capable of obtaining an index indicating how effective an intervention is on the person (i.e. how much the person's efficiency or productivity is increased by the application of an intervention). The index is objectively calculated on the basis of (i) the measured performance as exhibited by a person in executing a task, and (ii) on the basis of the estimated emotional state of the same person. Since also the emotional state can be objectively and accurately estimated, the index consequently provides an objective and reliable indication about the intervention effectiveness. The index may thus be used in computing systems for a variety of applications, like for instance monitoring effectiveness of interventions, conveniently indexing different interventions, selecting suitable interventions, etc.

### First Embodiment

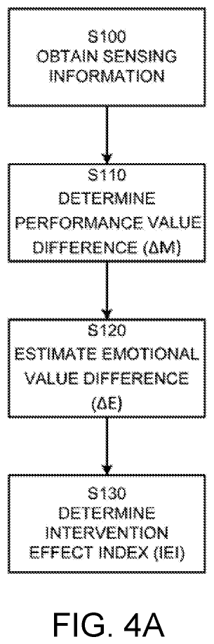

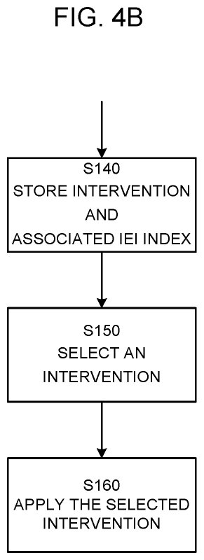

With reference to FIG. 4, a first embodiment will be described directed to a method for determining an intervention effect index WI for a person executing a task. An intervention is an excitation affecting the person. The intervention may be provided to the person (e.g. by providing a stimulus), in which case it directly affects the person, or provided to a system/device with which the person is interacting (when performing the task: e.g. by changing speed of operation of a manufacturing line machine) such that the intervention will indirectly affect the person. Examples will be further later discussed.

At step S100, sensing information is obtained by means of at least one sensor coupled to the person. The sensing information includes information relating to measurements taken by a sensor (as above discussed e.g. in relation to FIG. 2 or 3, or as later also illustrated) and, more in general, to any information relating to the person as captured by a suitable instrument including e.g. a camera, video camera, etc. The latter are therefore further examples of a sensors according to the present discussion. By coupled to the person it is herein meant that the sensor (in the sense of a sensing/capturing instrument) is within a range suitable for measuring or capturing the respective information. Therefore, the sensor may be at least partially engaged (i.e. at least in part of physical contact) with the person, or it may be in a suitable range with the person for the capturing to occur, e.g. a range allowing sounds, images, videos, any physiological signal, etc. of the person to be taken.

The sensing information includes first sensing information relating to performance (M) in executing the task, and second sensing information relating to an emotional state E of the person. For convenience, in the following, the first sensing information and second sensing information may also be referred to as performance related sensing information and, respectively, emotional related sensing information. Sensing information generally includes information measured or captured by means of a suitable sensor in the sense of any instrument capable of measuring or capturing information about a subject.

The performance M in executing the task indicates the degree of efficiency and/or accuracy in completing the task, or in other words how well and/or fast the task has been executed, or in further other words how successful the completion of the task is in relation to certain reference parameter(s). For example, the performance M can be obtained on the basis of a comparison between performance parameters (e.g. accuracy/quality of the result of the task, and/or speed/time required in completing the task, etc.) of the actually performed task and reference performance parameters (e.g. reference accuracy/quality, and/or reference speed/time required, respectively) of the same task (e.g. reference performance parameters of a task taken as reference task). In a specific non-limiting example, let us consider the case of a reference task being the production of one metallic component, wherein reference performance parameters include a mechanical tolerance of +−2% and a speed of production of 100 units/hour; when a worker executes the task of producing such metallic component, the actual performance can be obtained by comparing the actual tolerance (e.g. 5%) and actual speed (e.g. 50 pieces/hour) of production achieved by the worker against the respective reference parameters. In the example, the performance may be judged as low, and associated for instance a low value within a predetermined scale of values.

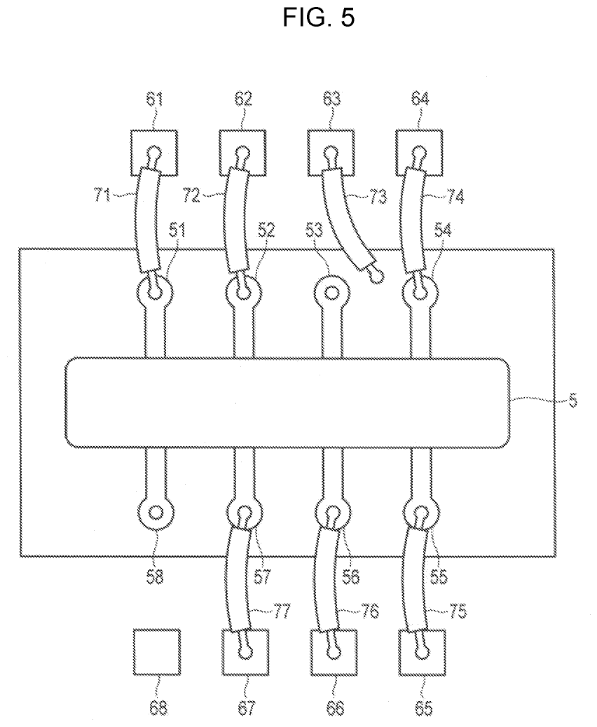

The first sensing information can be obtained by one or more suitable sensors, like for instance a camera (for still pictures and/or video) for capturing the worker while performing the task, a counter for measuring how many times the task is completed (preferably within a certain time interval), a timer for measuring how long it takes for the task to complete, etc. The sensed information can then be compared to reference values: for instance, the measured time is compared to a reference time, the measured count against a reference count, etc. How other information like a camera-captured image can be used can be explained with one example, wherein a worker is currently connecting parts. The image data about the operation results is as shown in FIG. 5, i.e. the figure represents the result of the task of connecting parts. In this example, the operation ends with a terminal 53 and a terminal 63 unsuccessfully connected using a lead 73, and a terminal 58 and a terminal 68 unconnected. A reference picture (not illustrated) would instead show that all parts are correctly connected; thus, by comparing (e.g. by means of an image elaboration process) the actual picture against the reference picture, it is possible to determine that the task has not been accurately completed, since it does not provide the intended result as indicated in the reference picture. Similar considerations apply to other types of captured information like a video, sound, etc.

The second sensing information relating to an emotional state is information that can be obtained from any sensor suitable for estimating an emotional state; examples of such sensors are given above, for instance in connection to FIGS. 2 and 3.

At step S110, a performance value difference (ΔM) is obtained on the basis of the first sensing information. The performance value difference (ΔM) corresponds to (or is indicative of) a variation between performance in executing the task before an intervention is applied and performance in executing the same task after the intervention is applied. The first sensing information is used in determining the performance before and after the intervention. For example, the performance variation may be calculated on the basis of the difference between the value of one performance related parameter (e.g. speed to complete task, accuracy of the result, etc.) before applying the intervention, and the value of the same performance related parameter after the intervention is applied. When taking the actual operation time (being the time required to complete the task) as an example of a performance related parameter, the difference between the actual operation time after and before the intervention is applied can be taken as the performance value difference (ΔM). In obtaining the ΔM, the value before the intervention can be subtracted to the value after the intervention, or vice versa. Also, an absolute value of the difference result may be taken. Further, ΔM need not be identical to the difference: it can be corrected by any factor. Still further, multiple performance related parameters may be considered, and combined in any way to determine the ΔM. For instance, differences between related parameters may be considered, and the obtained differences each multiplied by a certain factor can be combined together by addition to obtained ΔM as indicated by:

ΔM=a1×ΔM1+a2×ΔM2+ai×ΔMi+ . . . an×ΔMn,

wherein ΔMi relates to the difference between performance related parameter i (e.g. current operation time) before and after intervention, and ai is a respective corrective coefficient. More in general, ΔM is a function of one or more ΔMi, and can be expressed as: ΔM=f(ΔM1, ΔM2, . . . , ΔMi, . . . ΔMn).

At Step S120, an emotional value difference ΔE is estimated on the basis of the second sensing information. The emotional value difference ΔE indicates a variation between the emotional state of the person before the intervention is applied, and the emotional state of the person after the intervention is applied.

As anticipated, even if the emotional state cannot be directly measured, it can be reliably and objectively estimated on the basis of measurements made on the subject, see also the above discussion in relation to FIG. 1. Moreover, Japanese patent application JP2016-252368 filed on 27 Dec. 2017 as well as PCT application by same Applicant and filed by the same applicant as the present application which is one with reference/docket number 198 759 provides a detailed description of how a computing system can be configured and operated in order to obtain, autonomously by such computing system, an estimation of the person, wherein such estimation is objective and repeatable since it is systematically determined on the basis of objective measurements. Example of sensors suitable to collect measurement information for emotion estimation are above discussed with reference to FIGS. 2 and 3. The emotional value is thus determined on the basis of an emotional state Eb estimated before the intervention is applied, and an emotional state Ea after the intervention is applied. Once the two estimations Eb and Ea are estimated, the emotional variation ΔE can be obtained on their basis, e.g. as their difference, or its absolute value, optionally corrected by a predetermined factor. In general, ΔE is a function of Eb and Ea, i.e. ΔE=f(Eb, Ea).

At step S130, an intervention effect index IEI is determined on the basis of the performance value difference ΔM and the emotional value difference ΔE. The intervention effect index IEI provides an indication about the effectiveness of the intervention provided on the person. In other words, the IEI index indicates how successful the intervention has been in achieving the intended result of increasing efficiency of the person in performing a certain task. In further other words, the IEI index indicates how well or how far the intervention has managed to increasing efficiency. In general, any function taking the two inputs a ΔM value and a ΔE value into account is suitable for obtaining the IEI index, i.e. IEI=f(ΔM, ΔE).

The IEI index is an accurate value, since it takes into account not only the effect of the intervention on the performance of the person (ΔM), but also the effect of the intervention on the emotional state (ΔE) of the person; as such, the IEI index provides an overall accurate indication of how effective a given intervention is. As the inventors have recognized, in fact, it is important taking into account also the emotional state, since the actual effectiveness reached by the person depends not only on the performance in completing the task, but also on his/her emotional state (e.g. improved emotional state leads to: higher concentration in performing the task, which directly results into higher productivity/efficiency; to mid- or long-term improved mental state leading to higher quality in performing the task, etc.). If the emotional state would instead be neglected, it would not be possible to properly evaluate how effective the person really is in performing the task. Significantly, the emotional state is estimated, as in fact it would not be directly obtainable or detectable from the execution of the task of or from the result obtained when the task is completed. Further significantly, as above explained, the index IEI is obtained by means of repeatable and objective operations (determination of ΔM, estimation of ΔE), all based on objective and repeatable measurement. Thus, the IEI index is particularly suitable for a computing system intended for instance to monitor how effective an intervention is, or to select an effective intervention depending on circumstances, etc. In fact, if one would conceive evaluating the emotional state differently, e.g. by using an observer trained to judge emotional states from verbal and/or facial expressions of the worker (before and after intervention), one would rely on a system prone to errors, to subjective judgements, and importantly not suitable for being implementing in a computing environment. Also, even when using such an observer-based-solution, one would require complex and storage-consuming data structures. Using instead the above illustrated IEI index, it is possible providing an objectively defined data for each intervention, which can be easily and effectively used in a computer system without large resources.

Optionally, the method of the present embodiment comprises a step of storing, in a database, at least one of a plurality of intervention information with a respective intervention effect index (ΔP) determined from the determining step (S130). The intervention information may include information identifying and/or describing an intervention. Information describing an intervention includes parameters describing or identifying the intervention, like for instance information about the type of intervention (e.g. audio, video, and/or electrical stimulus, etc.), and/or the timing at which it should be preferably applied, and/or the intensity, etc. Information identifying an intervention may include an ID pointing to one amongst a set of interventions for which respective parameters are known. Thus, the storing step ensures that the calculated IEI index is associated in a database with the information identifying or describing the intervention. In this way, the determined IEI index is ready for possible optional uses as below described.

Optionally, the method further includes a step of selecting, from a database (e.g. the above described database), an intervention based on a predetermined intervention effect index, wherein the predetermined indicates a target (wished) intervention effect index to be selected. For example, once multiple IEI indexes (previously determined as in FIG. 4) are stored in a database, one of them can be selected having an IEI index value equal (also in the sense of approximately equal, e.g. plus/minus respective tolerances) to a target index value. In this way, the intervention associated to the target IEI index can be selected, according to which a certain degree of effectiveness in improving the person's performance can be excepted. In other words, an intervention suitable for obtaining a certain overall improvement (corresponding to the target IEI index value) of efficiency/productivity can be conveniently selected, so that it can be applied if and when needed (e.g. once selected, it may be applied right away, or only when the efficiency falls below a threshold). In this way, it is possible to objectively control which intervention to use when wanting to achieve a certain wished improvement.

Preferably, the method of the present embodiment includes a step of applying the selected intervention. The intervention may be applied to the person and/or to the system with which the person is interacting. The system may include one or more devices, and an example of the system is represented by a manufacturing line including one or more manufacturing machines with which the person interacts when performing the task. Other examples of the system include: a vehicle driven by the person; a healthcare supporting device, etc.

Optionally, in the method of the present embodiment, the task includes a manufacturing task within a manufacturing line, and the intervention includes at least one amongst an intervention provided to the person and an intervention provided to at least one component included in the manufacturing line. In the following, illustration is given of an example illustrating this optional variant of the present embodiment.

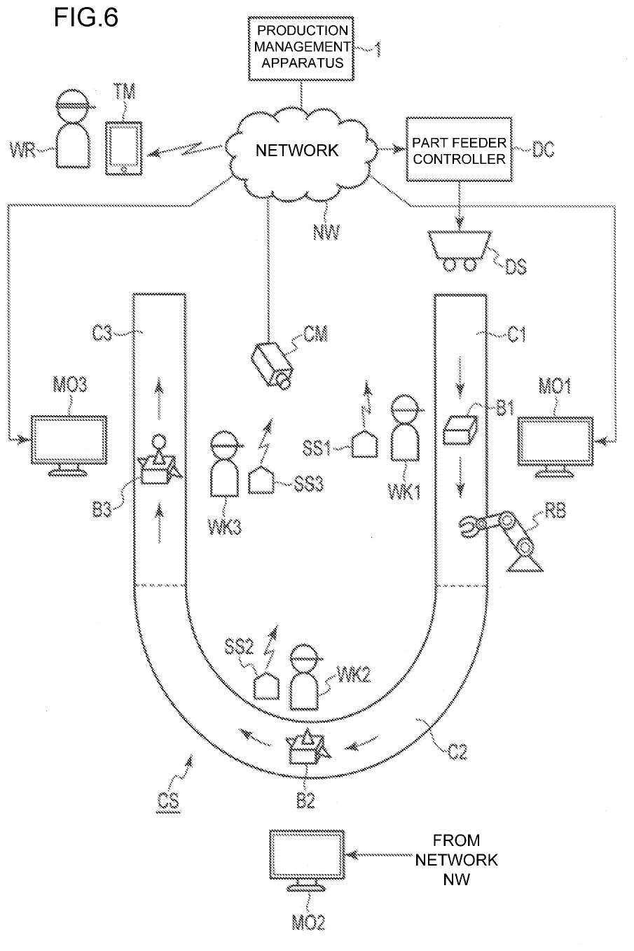

In the present example, the task includes a task performed on a manufacturing line like for instance making wire connections as illustrated in FIG. 5. The manufacturing line may comprise one or more components, and arranged in one or more sections, as for instance illustrated in FIG. 6. FIG. 6 shows an example cell production system, which includes a U-shaped production line CS. The production line CS includes, for example, three cells C1, C2, and C3 corresponding to different sections on the course of the products. Workers WK1, WK2, and WK3 are assigned to the cells C1, C2, and C3, respectively. In addition, a skilled leader WR is placed to supervise the overall operation on the production line CS. The leader WR has a portable information terminal TM, such as a smartphone or a tablet terminal. The portable information terminal TM is used to display information for managing the production operation provided to the leader WR. A part feeder DS and a part feeder controller DC are located most upstream of the production line CS. The part feeder DS feeds various parts for assembly onto the line CS at a specified rate in accordance with a feed instruction issued from the part feeder controller DC. Additionally, the cell C1, which is a predetermined cell in the production line CS, has a cooperative robot RB. In accordance with an instruction from the part feeder controller DC, the cooperative robot RB assembles a part into a product B1 in cooperation with the part feed rate. The cells C1, C2, and C3 in the production line CS have monitors MO1, MO2, and MO3, respectively. The monitors MO1, MO2, and MO3 are used to provide the workers WK1, WK2, and WK3 with instruction information about their operations and other messages. A work monitoring camera CM is installed above the production line CS. The work monitoring camera CM captures images to be used for checking the results of the production operations for the products B1, B2, and B3 performed by the workers WK1, WK2, and WK3 in the cells C1, C2, and C3 (e.g. the image captured by CM is as depicted in FIG. 5, and thus represent an example of first sensing information used to obtain the performance value difference ΔM). The numbers of monitors, sections, and workers, and the presence or absence of a leader may not be limited to those shown in FIG. 6. The production operation performed by each worker may also be monitored in any manner other than to use the work monitoring camera CM. For example, the sound, light, and vibrations representing the results of the production operation may be collected, and the collected information may be used to estimate the results of the production operation. To estimate the emotion of each of the workers WK1, WK2, and WK3, the workers WK1, WK2, and WK3 have input and measurement devices SS1, SS2, and SS3, respectively. The input and measurement devices SS1, SS2, and SS3 can be of any type or combination of sensors as illustrated with reference to FIGS. 2 and 3 (thus, devices SS1, SS2, SS3 are examples of instruments or sensors providing as output second sensing information, which are used for estimating the emotional state E and the variation ΔE in emotional states).

Going back to the example of FIGS. 5 and 6, the operation of making connections may for instance be performed by worker WK2. In this example of a task performed on a manufacturing line, the intervention may include an intervention provided (for instance, directly) to the person, and/or an intervention provided to at least one component included in the manufacturing line.

Examples of interventions provided to the person include any audio, and/or video, and/or text, and/or stimulus (e.g. electric signal for physiologically stimulating the person) provided to the person by suitable means or units like a display, speaker, electric stimulating device, etc. For instance, an audiovisual message (drawing the worker attention to certain technical points of the task, to take a rest, etc.) may be delivered to a portable device of the worker, or to the monitor MO2 easily visible to the worker WK2, or to another terminal available on the production line or to another person present on the production line; an electric stimulating signal may be conveyed to the worker by a physiologically suitable device coupled to the person.

As examples of intervention provided to at least one component included in the manufacturing line is the following: a line controller (not illustrated) transmits a speed change command to the part feeder controller DC to reduce the rate of feeding parts to the production line CS (other examples: a command to change speed of functioning and/or movement of any component of the line, like a tooling machine, etc.). This command lowers the rate of feeding parts from the part feeder DS to the line, as controlled by the part feeder controller DC. In this manner, the speed of the production line CS is adjusted as provided by the predetermined or selected intervention; the IEI index stored in the database indicates the degree of productivity increase provided by such a type of intervention on worker WK2, or how much the intervention contributes to an increase in productivity. It is herein noted, as found by the inventors, that a decrease in operating speed of the line does not necessarily mean an overall lower productivity. In fact, there are circumstances wherein a line working at reduced speed may lead to overall higher productivity: for instance, the lower speed may allow the worker, presently under a certain emotional state, to better execute the task such that the overall yield is increased when compared to line operated at a quicker speed for the same worker under the same mental state. Applying the intervention allows thus to increase the overall productivity; this fact is objectively expressed by the IEI index, which therefore allows to better control the productivity, or to better monitor the productivity, or to better predict the productivity.

The interventions above (as well as below) described can be identified by an ID identifying them amongst a set of predefined interventions, or described by parameters.

In the following, illustration is given of an example, further according to the first embodiment, of a task including an operation for driving a vehicle, and of an intervention provided on the driver and/or on at least a component of the vehicle.

Optionally, in the method of the present embodiment, the task includes an operation for driving a vehicle, and the intervention includes at least one amongst an intervention directly provided to the person (preferably when driving the vehicle) and an intervention provided to at least one component included in the vehicle. The operation for driving a vehicle includes any action that a driver may undertake when conducting a vehicle, including any supervising action in case the vehicle is equipped with an automatic or semi-automatic driving system. In the following, illustration is given of an example illustrating this optional (vehicle-related) variant of the present embodiment.

In the present example, the performance M indicates how correctly the driving task is executed, which can be determined e.g. by measuring certain driving parameters like how correctly the vehicle follows certain predetermined routes (e.g. comparing how smoothly the actual driving route correspond to an ideal route obtained from a map), how smooth the control of the vehicle is (e.g. whether or how often any sudden change of direction occurs), the degree of the driver recognizing an obstacle, etc. Suitable sensors could be provided, like for instance: a camera similar to (or the same as) the CM camera above described, conveniently installed to point inside and/or outside the vehicle to capture the driven route, the driving pattern, and/or the driver's movements; positioning measurement systems, e.g. to determine the driven route; vehicle speed sensors; vehicle inertial systems for obtaining information on current driving parameters, etc. The information provided by the sensors can also be used, according to other examples, to compare the distance covered over a certain period and an expected distance for a given period, or to determine whether in reaching two points a certain route has been followed compared to predetermined available routes, etc.: the obtained values provide also a measure of the performance M in performing a driving task. When such measurements are performed before and after an intervention, a performance value difference ΔM can be determined. The sensors (and information thereby produced by their sensing or capturing) are thus examples of sensors capable of providing first sensing information, on the basis of which the performance variation ΔM can be determined.

The driver emotion E can be estimated as above illustrated, and by means of suitable sensors also as above illustrated. Accordingly, also the emotion value difference ΔE can be estimated.

The intervention provided according to this example includes a driving assistance provided to the person (e.g. driver) and/or the vehicle. For example, driving assistance provided to the vehicle may include an active control of the vehicle by an assisting unit during driving, wherein the assisting unit may act on components of the vehicle like brakes, accelerator, and/or steering wheel, for instance in order to taking over for instance control of the vehicle, and/or stopping the same. Examples of the driving assistance provided to the driver include a feedback during driving, including a message (audio, video, and/or text; preferably audio) to the driver suggesting to make a stop and take a rest, or suggesting/guiding on how to better execute a certain task (e.g. when approaching a specific type of junction, etc.). Another example of driving assistance feedback is represented by a sound, melody, music, or audio message in general, that can for instance alert or prevent the driver from entering into any hazardous situation. In this example, the IEI index provides an indication of how much the driving performance can be increased for the person when applying a certain intervention. Thus, the overall driving performance can be better monitored, predicted, or further improved (e.g. by selecting an intervention, or driving assistance feedback in the above example).

Optionally, in the method of the present embodiment, the task includes an activity performed by the person, and the intervention includes a healthcare support feedback provided to the person, preferably by means of a healthcare supporting apparatus. Preferably, the activity includes a physical and/or intellectual activity or exercise of the subject. In the following, illustration is given of an example illustrating this optional (healthcare-related) variant of the present embodiment.

In the present example, the performance M indicates how well the activity is performed, for instance how well a physical and/or intellectual exercise is completed compared to a reference activity/exercise, and/or how quickly the exercise is completed. As a non-limiting example, the exercise includes a training exercise to improve or maintain a person cognitive ability and/or memory, wherein the cognitive ability may be for example a cognitive ability of performing a manufacturing task, a driving task, a task aimed at improving health conditions etc. In other words, in an example, the exercise may be a training exercise aimed at improving the ability to improve performing a certain (e.g. manufacturing, driving, etc.) task. The performance M can be obtained from: for instance, by determining how straight and balanced the person's body position is when walking, running or sitting (e.g. the actual position being compared over predetermined patterns); how smoothly certain movements are made over predetermined patterns; measuring the distance covered on foot over an expected distance; measuring the time for accomplishing a task over a predetermined time (e.g. completing a housecleaning or hobby-related operation, number of such operations performed in an hour or day), etc. The above information can be obtained for instance by comparing an image (obtained e.g. via a camera like the camera CM above described, which captures the subject while performing the activity) with a predetermined pattern, or by making other suitable measurements and comparing the same with predetermined values and/or pattern of values. Also, it is possible considering the performance M as related to how accurately and/or quickly certain intellectual exercises are executed, which can be measured by whether the outcome of the result is correct or not, and by the time required in producing the result.

The above discussed measurement results are examples of the first sensing information. They can be collected by a healthcare supporting device coupled to the person, i.e. the device can be in at least partial physical contact with the person or within a range (also without physical contact) such that measurements can be taken on the subject.

By making measurements/captures before and after an intervention is applied, it is possible to determine the performance value difference ΔM.

The person's emotion E can be estimated as above illustrated, and by means of suitable sensors also as above illustrated. Accordingly, also the emotion value difference ΔE can be estimated.

Once the ΔM and ΔE are obtained, the IEI index can be determined as also above illustrated. The IEI index indicates to which degree an intervention can improve the person capabilities of performing a certain activity, and is therefore an objective index of how the intervention can improve health conditions. It becomes thus possible to objectively monitor and predict health conditions provided by a certain intervention, or select an appropriate intervention for improvement the health conditions of the person.

Optionally, the method of the present embodiment includes a step of monitoring effectiveness of an intervention on the basis of the determined intervention effect index. In fact, it is possible to objectively establish the degree of effectiveness when a certain intervention is applied to a person; since the monitoring is based on objective parameters, the monitoring becomes reliable (since it is repeatable), objective and accurate.

Optionally, in the method of the present embodiment, the step of determining (S110) comprises determining a first performance value Mb and a second performance value Ma. The first performance value Mb is a value indicating the performance in executing the task based on the first sensing information obtained before the intervention is applied. The second performance value Ma is a value indicating the performance in executing the task based on the first sensing information obtained after the intervention is applied. In other words, the values Ma and Mb are the performance after and before the intervention is applied, and are obtained on the basis of measurements made at two different time points, namely after and before the intervention is applied. Before and after also include the case wherein one of the measurements used for determining one of them is taken while the intervention is applied; it is however preferable to maintain a certain time separation between the point in time when a measurement is made (either before or after the intervention) and the point in time when the intervention is applied. Further, the performance value difference (ΔM) is determined on the basis on the difference between said first performance value Mb and said second performance value Ma.

As above indicated, a database can be optionally foreseen, which stores at least one of a plurality of intervention information with a respective intervention effect index (ΔP). Further optionally, the method of the present embodiment includes determining an intervention effect index value in correspondence of a person; and selecting, from the database, an intervention to be applied to a person based on the determined intervention effect index value. In other words, it may be established that for a certain person at a given point in time, a certain IEI index is appropriate, for instance because the productivity of that person wants to be increased by a certain amount. Starting from the desired increase, an IEI index is determined, which therefore indicates the degree of potential productivity increase reachable by that person thanks to the intervention. The intervention corresponding to such IEI index is then taken from the database.

Optionally, in the method of the present embodiment, the database further stores attributes of a person in association with the intervention and with the respective effect index, and the method comprises a step of selecting an intervention on the basis of at least one of the stored attributes. The attributes may be stored at the same time of storing the intervention information and the respective IEI index, with attribute values corresponding to the person to which the intervention has been applied. For example, the database may include a list of one or more interventions each associated with a respective IEI index and with information like age, and/or sex, and/or experience of a worker, and/or type of training received in the past, etc. When productivity of worker WKx wants to be increased, it is determined that a certain IEI index is necessary; it may occur that different interventions having the same or a similar IEI index are present in the database; it may thus be advantageous selecting, from the database, an intervention associated to attribute information as close as possible to the attribute information of worker WKx. In this way, it is possible extending the application of stored data also to other workers for which no IEI index has been already calculated, or for which only few interventions are stored.

### Second Embodiment

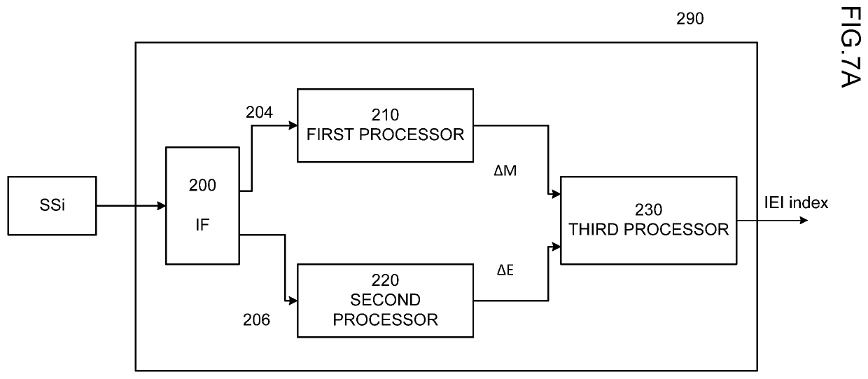

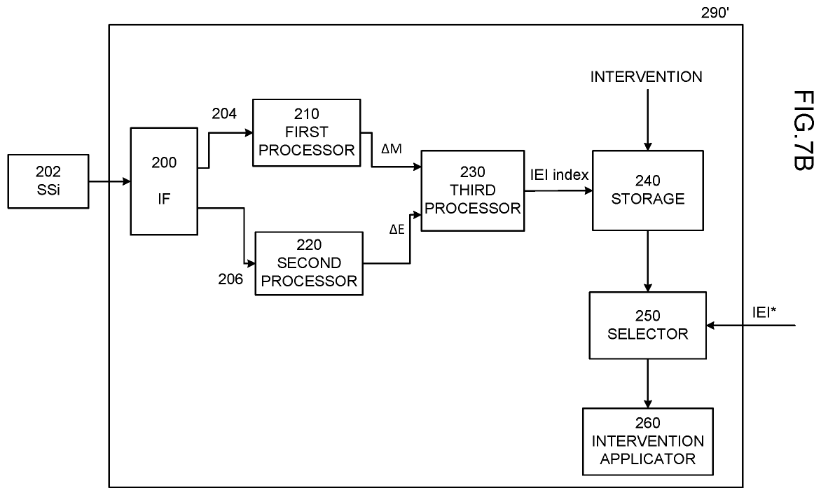

With reference to FIG. 7A, description will be made of a second embodiment directed to an apparatus 290 for determining an intervention effect index IEI for a person executing a task. The apparatus can be regarded as an apparatus that can be realized by any combination of hardware and/or software, and can be either localized into one device or distributed over multiple devices interconnected through a network. In the following, the term apparatus is also interchangeably used with the term entity, as it is not limited to a centralized implementation in a single device. Further in the following, the main features of the entity are described, when noting that all considerations and other features described above in relation to the method of the first embodiment are equally applicable to the present and other embodiments, such that they will not be repeated.

The entity comprises an interface 200, a first processor 210, a second processor 220 and a third processor 230. The interface 200 is configured to obtain sensing information by means of one or more sensors, generally depicted as SSi, coupled to the person. Any type of interface either wired (e.g. USB, FireWire, etc.) or wireless (e.g. WLAN, Bluetooth, etc.) is suitable for collecting information measured or captured by any of sensors SSi. The sensor(s) SSi are not necessarily part of the entity 290, as shown in the figure. The sensor(s) SSi provide the interface 200 with sensing information including first sensing information and the second sensing information, which in the figure are forwarded as the first sensing information 204 to the first processor and as second sensing information 206 to the second processor. It is noted that even though first to third processors are herein described, they can be conveniently combined into one single processor realized by any combination of hardware and/or software. Similarly, the first 204 and second 206 sensing information are only schematically separated, and can be in fact be provided altogether to the processor. Thus, the figure as well as the following illustration is in no way limiting. The first sensing information 204 relates to performance in executing the task and the second sensing information 206 relates to an emotional state.

The first processor 210 is configured to determine, on the basis of the first sensing information 204, a performance value difference ΔM indicating a variation between performance in executing the task before an intervention is applied, and performance in executing the task after the intervention is applied. The intervention represents an excitation affecting the person. The second processor 220 is configured to estimate, on the basis of the second sensing information 206, an emotional value difference ΔE indicative of a variation between emotional states before and after the intervention is applied. The third processor 230 is configured to determine the intervention effect index IEI on the basis of the performance value difference ΔM and of the emotional value difference ΔE. The intervention effect index IEI represents an indication of effectiveness of the intervention on the person.

Reference will now also be made to FIG. 7B, showing an entity 290′ representing optional different configurations of the entity 290 of FIG. 7A. Same reference signs in FIGS. 7B and 7A refer to same features, such that reference is made to the above description of FIG. 7A. The entity 290′ may optionally comprise a storage unit 240 configured to store at least one of a plurality of interventions information in association with a respective intervention effect index preferably determined by the third processor 230. Further, the entity 290′ may optionally comprise a selector 250 configured to select, from a database, an intervention based on a predetermined intervention effect index. The selection can be made from the storage 240 including such a database, or from an external device including such a database. In fact, as also above explained, the storage unit 240 is only optional and does not need to be necessarily provided within the entity. Any type of database or way of storing data in association with each other (including a table) can be used. Optionally, the entity may include an intervention applicator 260 configured to apply a certain intervention, preferably the intervention selected for instance by their selector 250. In FIG. 7B, the applicator 260 is illustrated as part of the entity 290′. However, the applicator 260 may be externally provided into a portable device, which can be coupled to the person. In such case, the selector 250 may transmit information to the applicator indicating which intervention to apply, for instance the transmitted information may include the intervention information also above discussed (namely, and identifier and/or relevant parameters for describing the intervention).

In one possible optional configuration of the second embodiment, the entity 290 (or 290′) may be used for determining an intervention effect index IEI, in which case the task executed by the person includes a manufacturing task within a manufacturing line. In this case, the intervention includes an intervention provided to the person and/or an intervention provided to one or more components included in the manufacturing line.

In one further possible optional configuration of the second embodiment, the entity 290 (290′) may be used in a driving assistance system providing assistance to a person (the driver) in driving a vehicle. In this case, the task includes an operation for driving a vehicle, and the intervention includes an intervention directly provided to the person and/or an intervention provided to at least one component included in the vehicle. The entity may be provided in different configurations: within the vehicle, or outside of the vehicle (e.g. at least some of the operations performed by the processors are performed on a cloud or server). In the latter case, the entity is in communication with components at the vehicle, like for instance sensors SSi provided at the vehicle. With reference to FIG. 7B, when the entity is developed outside the vehicle, the intervention applicator 260 is in (preferably wireless) communication with the entity.

In one a further possible optional configuration of the present embodiment, the entity 290 (290′) may be used in a healthcare support system for providing a healthcare support to a person. In this case, the task includes an activity performed by the person, and the intervention includes a healthcare support feedback provided to the person. In this configuration, the entity may be provided within a portable device coupled to the person. In an alternative configuration, the entity may be provided remotely from the person (for instance on a cloud or a server) which is in communication with the sensor(s) SSi and optionally a selector locally provided in a device coupled to the person (it is noted that coupled is not necessarily imply a physical contact between a device and a person).

Optionally, the entity of the present embodiment may include a monitoring unit configured to monitor the effectiveness of an intervention on the basis of the determined intervention effect index. In one configuration, the monitoring unit may replace the selector units 250, or be provided together with the selector 250; in the latter case, the entity is capable of performing both monitoring and application of interventions.

The entity may further be configured to operate according to the details illustrated in the first embodiment, to which reference is made.

### Third Embodiment

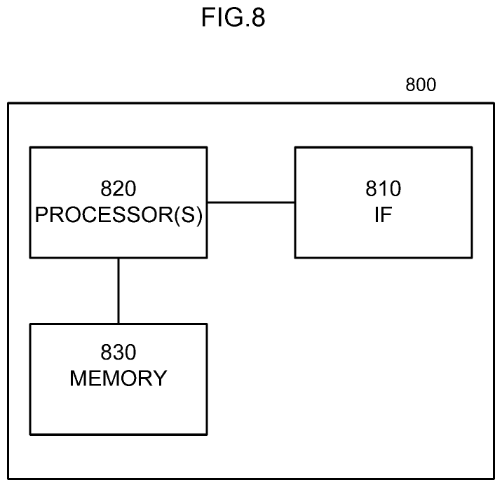

A third embodiment relates to a computer program including instructions which, when executed on a computer, causes the computer to execute any of the steps or any combination of the steps disclosed for instance with reference to the first embodiment and/or with reference to other embodiments and examples herein described. FIG. 8 illustrates a configuration for such a computer 800, including a processor 820, an interface (IF) 810, and a memory 830. In one configuration, a memory 830 may include instructions necessary for executing the method steps, as well as for instance data necessary to or produced during the execution of the instructions. At the same time, the database may be externally provided to the computer 800, and accessed via the interface 810. The interface 810 is capable of communicating to sensors, to an applicator when present, and to an external database when present, as well as to any other entity or network. The possessor 820 is any type of processor capable of executing the mentioned instructions. The computer 800 is schematically illustrated in one block unit 800; however, it can be realised in one localized apparatus, or in a plurality of apparatuses connected through a network. Furthermore, the computer 800 can be realised by any combination of software and/or hardware. As also above discussed, the processor can be a single localized unit, or may be implemented into more processors.

The instructions can be stored on a medium, including any type of non-volatile memory, or may be carried on a signal for being executed for instance by a remote entity.

### Fourth Embodiment

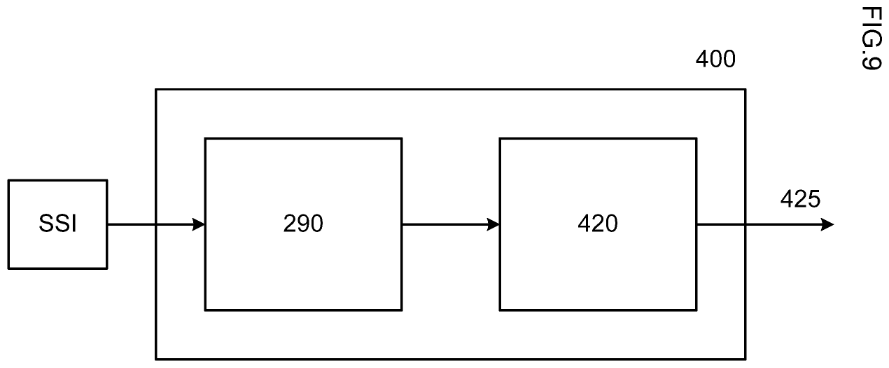

According to a further embodiment, it is provided a system 400 as also depicted in FIG. 9. The system 400 includes an apparatus (entity) like in the second embodiment (e.g. an apparatus (entity) 290 as in FIG. 4A, or 290′ as in FIG. 4B) and an intervention applicator 420. The intervention applicator 420 is preferably portable (so that it can be coupled to the person also when the person is moving), but not necessarily (in which case it will be coupled to the person when the person is within range of interaction with the applicator). The system 400 can receive inputs for instance from sensor(s) like SSi, and provide output 425. The output 425 can include the intervention to be provided to the person.

### OTHER EMBODIMENTS AND EXAMPLES

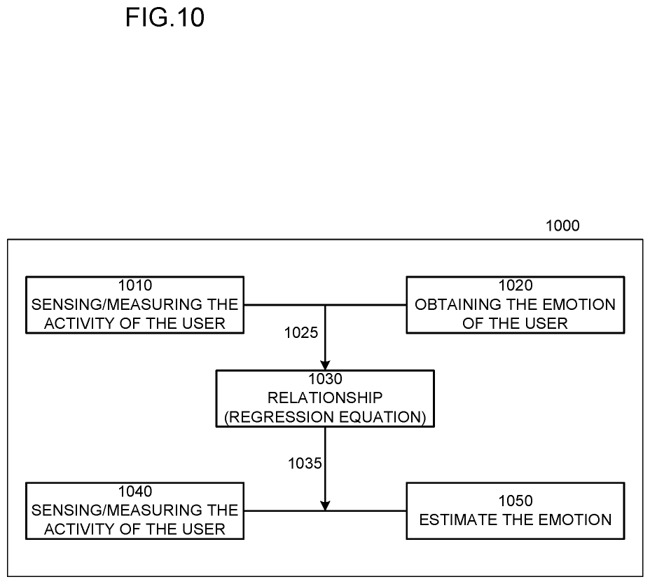

It is noted that the estimation of the emotional value can be obtained for instance as disclosed in related Japanese patent application JP2016-252368 and respective PCT application as above referred (reference/docket number 198 759). However, any other method is applicable, as long as it is capable of providing an estimation of the emotional state on the basis of objective measurements. Therefore, step S120 as above illustrated may receive, as input, the emotional states calculated according to the method disclosed in the mentioned related application, once before the intervention is applied and once after the intervention is applied. In an alternative configuration, step S120 may comprise one or all steps disclosed in related application. Similar considerations apply to the second processor 220 of the second embodiment. FIG. 10 illustrates one way of estimating the emotional state, as for instance detailed in Japanese patent application JP2016-252368 and respective PCT above referred (reference/docket number 198 759). Namely, a learning process is performed on data 1010 and data 1020, which result in data 1025 representing a relationship between activities and emotions. The data 1010 relate to measurement captured in relation to activities performed by one or more persons; the data 1020 relates to information about the person emotion when performing a corresponding activity (such emotional state can be obtained by means of suitable sensors, for instance highly accurate sensors as described in relation to FIGS. 1 to 3). Once the learning data 1025 has been obtained, during operation, a current emotion of a person can be estimated (not directly measured), (e.g. by unit 150 configured to estimate an emotion) starting from measurements about an current activity performed by the same person (e.g. which measurements are acquired by a unit 1040 configured to sense/measure the activity of the person/user), and the learning data 1025. The learning data 1025 may also be obtained by means of a regression equation, and represented by a relationship. Also, the learning data 1025 may be obtained and/or stored in a unit 1030 capable of obtaining and/or storing such relationship.

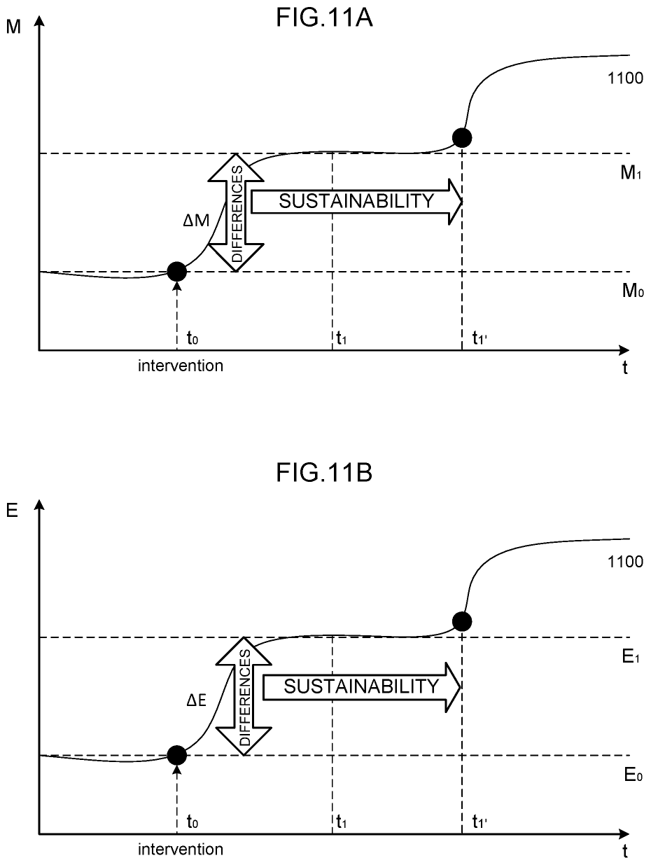

FIG. 11 is used to schematically illustrate, in a simplified manner, some features above discussed. In particular, FIG. 11A shows an example of how the performance of a person executing a task varies over time. For example, assuming that the intervention is applied at time to, the performance increments thereafter by an amount corresponding to ΔM. The increase may not be instantaneous, such that it is preferable waiting for a certain interval, before taking a measurement of performance after the intervention is applied. In the figure, an example of such a time is represented by t1. The value t1 can be predetermined, and chosen depending on circumstances or application (e.g. for a vehicle application this may be shorter than for a manufacturing line application). Also, the value t1 may be dynamically determined: for instance, it is a point in time following a predetermined time interval during which the performance has been approximately constant at an increased value (i.e. to exclude short peaks of performance probably not related to the intervention). At time t1′ the performance starts changing again, indicating that the performance is likely not anymore directly linked to the intervention at time t0. The time interval ΔT, being the difference between t1′ and t0 represents the sustainability of the intervention, i.e. until when it lasts after the intervention is applies. Due to a transient immediately following t0, ΔT does not necessarily mean that the performance is equal to the same increased value throughout the interval. Also, the figure shows a constant increase for a certain time value; it goes without saying that there may be fluctuations and that this may be taken into account with appropriate thresholds.

As already stated, the value M is a value directly obtained (i.e., not estimated) by measurements. It can be said that M gives an indication of human's output, and represents activities and/or the person current state.

The output to be measured may be selected depending on the field of application, like e.g. manufacturing support, driver monitoring, driving assistance, healthcare monitoring, healthcare support, etc.). Also, it can be said that M is an output which is used to assess the user's performance and/or skill and/or ability. Interventions may be provided in order to enhance and improve the person performance/skill/ability. These are non-limiting examples of how to measure M, depending for instance on the field of application:

| Field of application | M |
|---|---|
| Manufacturing support | Operation time, speed, error rate, etc. |
| Driving | Response time, correctness in following a route |
| Healthcare | Heart rate |

FIG. 11B shows how the emotional state E varies over time. Also in this example, the emotional state value E increases after the intervention is applied at time t0, and lasts until t1′ (in the sense that from t1′ the performance may not be any more directly linked to the intervention at time t0). Similar consideration made with reference to FIG. 11A apply also here.

The performance and the emotion together influence the overall efficiency or productivity of a person, which can be varied by means of an intervention. Thus, the IEI index provides an accurate indication to how effective an intervention is. As such, the IEI index is capable of indicating how suitable an intervention is for a certain application (manufacturing, driving, healthcare, etc.); further, the IEI is capable of more accurately assessing the provided intervention (e.g. the objective of an intervention is to improve the user's performance/skill/ability itself; thus, the IEI index facilitates such assessment in an objective way).

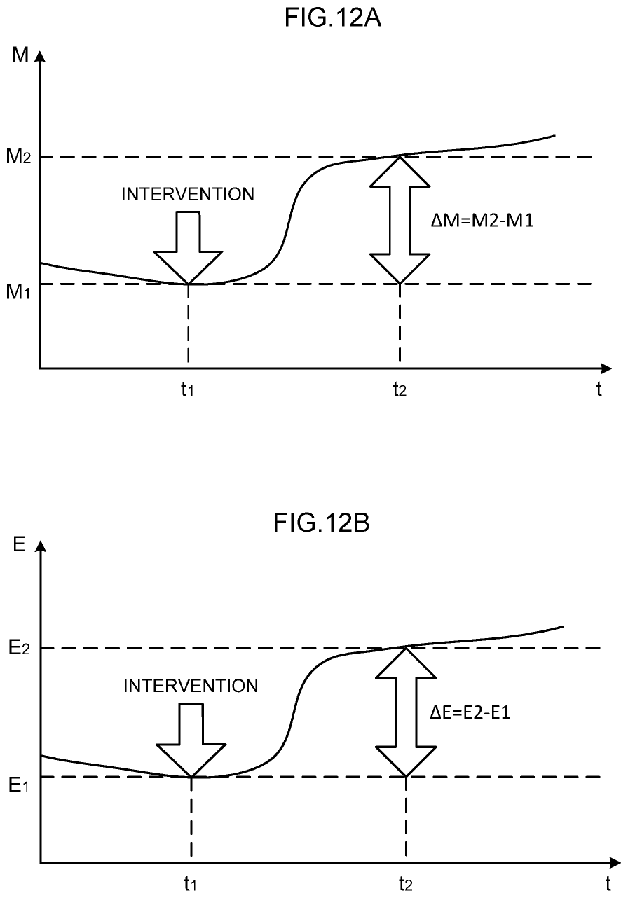

FIGS. 12A and 12B are further illustrations of how the performance M and the emotional state E vary after the intervention is applied at time t1. The IEI index, as already described, is a general function of ΔM and ΔE, i.e.

IEI=f(ΔM,ΔE).

In one example, the IEI index may be obtained by integrating ΔM and ΔE using respective weights α and β:

IEI=α*ΔM+β*ΔE

Further, ΔM and ΔE may be absolute values. Preferably, ΔM and ΔE are normalized values so that they become comparable to each other. For example:

ΔM=ΔM_raw data/ΔM_reference,

ΔE=ΔE_raw data/ΔE_reference

wherein ΔM reference and ΔE reference may be set in advance as average values.

The above weights α and β can be set in a variety of ways. Non-limiting examples are:
The weights α, β may have the same value (e.g., α=1, β=1; or α=2, β=2)
The weights α, β may be determined based on the importance of M and E in executing the task (e.g. depending on the field of application), such that one having higher importance has the larger weight.
The weights α, β may be determined based on the improvement of M and E by the intervention, such that one having higher positive variation (which indicates improvement) has the larger weight.
When one of M and E degrades after the intervention (i.e., the difference is negative value), then the weights may be determined such that the degraded one has larger weight (to estimate the smaller IEI index for the intervention which does not improve M or E).

As above discussed, it is possible to select an intervention, for instance from a database. For instance, the intervention can be selected as one having a target IEI value, i.e. as one intervention which achieves a certain degree of improvement. The value of the target IEI can be absolute (IEI_target), or relative (ΔIEI_target) to a current IEI value.

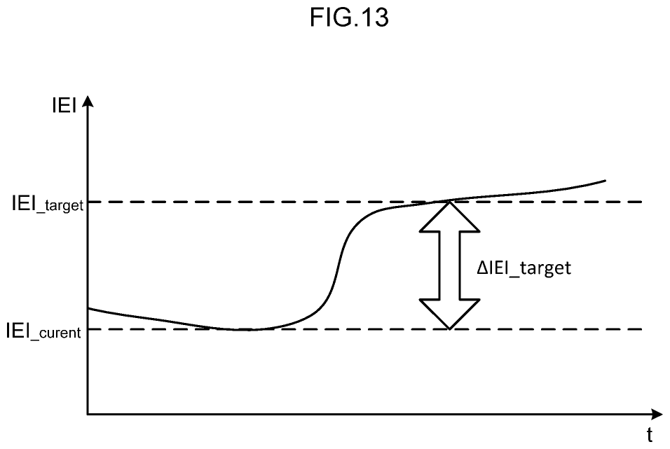

Referring to the example of FIG. 13, it can be seen that the current IEI index for the person corresponds to the IEI_current value on the vertical axis; for instance, IEI_current is determined on the basis of another intervention applied before the current time t1, or on the basis of two measurements for each of M and E performed at two different points in time (close to the current time t1) so as to calculate IEI_current. When it is wished to obtain an improvement of effectiveness equal at least to ΔIEI_target, then it is needed to select from a database an intervention having IEI_target that satisfies the following:

ΔIEI_target=IEI_target−IEI_current

In another example, it is determined, from the database storing interventions, the intervention(s) having ΔIEI equal to or larger than ΔIEI_target corresponding to the required increase. When more than one intervention is accordingly determined, one amongst them can be selected based for instance on one of the following examples:

[ex.1] the intervention having maximum ΔIEI is selected

[ex.2] the intervention having ΔIEI within a predetermined range (e.g. when ΔIEI_target=80, an intervention with 80≤ΔIEI≤100 is selected).

Moreover, when two or more interventions are extracted, all of them having the same ΔIEI, the following condition [ex.3] can be used to select one intervention:

[ex.3] the intervention that increases both M and E (i.e., M2−M1/E2−E1 is not negative). Furthermore, when two or more interventions are extracted having the same ΔIEI, the intervention having maximum M+E can be selected.

[ex.4] An intervention can be selected that matches the subject's attribute such as sex, age, country, or the subject's request (input in advance)

Further, when two or more interventions are extracted, conditions according to above ex.2 or ex. 3 can be used to determine one intervention.

In general, the above conditions can be combined together in any manner as appropriate according to circumstances.

As an illustrative example, a database storing multiple interventions can be as follows (represented in the form of a table only for facilitating understanding):

| | | | | Subject type/ |
|---|---|---|---|---|
| ID | Contents/Means | Timing | Index | attribute |
| 1 | Showing video | Middle of the | ΔIEI, ΔM, | Ages |
| | recording the | work | ΔE | Male/Female |
| | expert's operation | | | Experience |
| 2 | Displaying positive | ΔIEI < Th | ΔIEI, ΔM, | |
| | and negative | End of the work | ΔE | |
| | message | | | |
| 3 | Displaying positive | ΔIEI < Th | ΔIEI, ΔM, | |
| | message | End of the work | ΔE | |
| 4 | Displaying negative | ΔIEI < Th | ΔIEI, ΔM, | |
| | message | Middel of the | ΔE | |
| | | work | | |
| 5 | Providing vibration | ΔIEI < Th | ΔIEI, ΔM, | |
| | | | ΔE | |
| 6 | Changing the light | ΔIEI < Th | ΔIEI, ΔM, | |
| | | Morning/Afternoon/ | ΔE | |
| | | Evening | | |
| 7 | Outputting the | ΔIEI < Th | ΔIEI, ΔM, | |
| | music | | ΔE | |
| 8 | . . . | | | |

Starting from left, the first column illustrates an ID, which allows to identify the intervention. The second column specifies the type of intervention, and preferably contains details for creating the stimulus (like a link to the message or video or excitation signal composition information, or the intervention itself). The third column indicates a timing at which the intervention is to be preferably applied, wherein Th indicates a threshold (different thresholds can be specified). The fourth column contains at least one index or a combination of index, like for instance the IEI (not illustrated), and/or ΔIEI, and/or ΔM, and/or ΔE. Depending on how many and which indexes are stored, different selecting conditions can be applied, see above. The last column includes attributes of the person to which the intervention has been applied, wherein age, sex, experience are non-limiting examples combinable in any manner. The attribute information may be useful in determining an intervention for a subject, by searching for interventions applied to subjects having similar attributes.

In the methods and examples herein described, steps are mentioned like obtaining, determining, estimating, storing, applying, monitoring, selecting, etc. It is however noted that such steps (or any combination of them) may also be caused or induced by a remote device, like for instance by a client computer or a portable terminal, on another device (like for instance a server, localized or distributed) that correspondingly performs the actual step. Thus, the mentioned steps are to be understood also as causing to obtain, causing to determine, causing to estimate, causing to store, causing to apply, causing to monitor, causing to select, etc. such that any of their combination can be caused or induced by a device remote to the device actually performing the respective step.

The above embodiments are only illustrative, and in fact the present invention is also applicable to other fields like construction machinery, power generation equipment, electrical transformers, medical devices operated by the person, as well as control systems for various plants, airplanes, or trains.

It will be apparent to those skilled in the art that various modifications and variations can be made in the entities, methods, systems, computer programs, medium and signals (carrying instructions for executing the program) of the invention as well as in the construction of this invention without departing from the scope or spirit of the invention. Also, the entities have been described in terms of processors, memory (or storage unit), etc.; the same terms can be replaced by respective means (e.g. processing means, storing means, etc.) as in fact the invention is not limited to a processor, a memory, etc. and can in fact be implemented by any suitable means for performing the same functions, or steps as also described in the above embodiments and examples. The invention has been described in relation to particular embodiments and examples which are intended in all aspects to be illustrative rather than restrictive. Those skilled in the art will appreciate that many different combinations of hardware, software and firmware will be suitable for practicing the present invention, which scope and spirit is defined by the following claims.

1. A method for determining an intervention effect index for a person executing a task, the method comprising steps of:
obtaining sensing information by means of at least one sensor coupled to the person, wherein the sensing information includes first sensing information relating to performance in executing the task and second sensing information relating to an emotional state;
determining, on the basis of said first sensing information, a performance value difference indicating a variation between performance in executing the task before an intervention is applied and performance in executing the task after the intervention is applied, the intervention representing an excitation affecting the person;
estimating, on the basis of said second sensing information, an emotional value difference indicative of a variation between emotional states before and after the intervention is applied;
determining the intervention effect index on the basis of said performance value difference and said emotional value difference, the intervention effect index representing an indication on effectiveness of the intervention on the person.

2. The method according to claim 1, further comprising the step of:
storing, in a database, at least one of a plurality of intervention information with a respective intervention effect index determined from said determining step for determining the intervention effect index.

3. The method according to claim 1, further comprising:
selecting, from a database, an intervention based on a predetermined intervention effect index.

4. The method according to claim 3, further including a step of applying the selected intervention.

5. The method according to claim 1, wherein the task includes a manufacturing task within a manufacturing line, and the intervention includes at least one amongst an intervention provided to the person and an intervention provided to at least one component included in the manufacturing line.

6. The method according to claim 1, wherein the task includes an operation for driving a vehicle, and the intervention includes at least one amongst an intervention directly provided to the person and an intervention provided to at least one component included in the vehicle.

7. The method according to claim 1, wherein the task includes an activity performed by the person, and the intervention includes a healthcare support feedback provided to the person.

8. The method according to claim 1, further including:
a step of monitoring effectiveness of an intervention on the basis of the determined intervention effect index.

9. The method according to claim 1, wherein the step of determining the performance value difference comprises:
determining a first performance value in executing the task based on said first sensing information obtained before the intervention is applied,
determining a second performance value in executing the task based on said first sensing information obtained after the intervention is applied,
determining said performance value difference based on the difference between said first performance value and said second performance value.

10. The method according to claim 2, further comprising the steps of:
determining an intervention effect index value in correspondence of a person;
selecting, from the database, an intervention to be applied to a person based on the determined intervention effect index value.

11. The method of claim 2, wherein the database further stores attributes of a person in association with said intervention and said respective intervention effect index, the method comprising selecting an intervention on the basis of at least one of said stored attributes.

12. An apparatus for determining an intervention effect index for a person executing a task, the apparatus comprising:
an interface configured to obtain sensing information by means of at least one sensor coupled to the person, wherein the sensing information includes first sensing information relating to performance in executing the task and second sensing information relating to an emotional state;
a first processor configured to determine, on the basis of said first sensing information, a performance value difference indicating a variation between performance in executing the task before an intervention is applied and performance in executing the task after the intervention is applied, the intervention representing an excitation affecting the person;
a second processor configured to estimate, on the basis of said second sensing information, an emotional value difference indicative of a variation between emotional states before and after the intervention is applied;
a third processor configured to determine the intervention effect index on the basis of said performance value difference and said emotional value difference, the intervention effect index representing an indication on effectiveness of the intervention on the person.

13. The apparatus according to claim 12, further comprising a storage unit configured to store at least one of a plurality of intervention information with a respective intervention effect index determined by the third processor.

14. The apparatus according to claim 12 or 13, further comprising:
a selector configured to select, from a database, an intervention based on a predetermined intervention effect index.

15. The apparatus according to claim 14, further comprising an intervention applicator configured to apply the selected intervention.

16. The apparatus according to claim 12, wherein the task includes a manufacturing task within a manufacturing line, and the intervention includes at least one amongst an intervention provided to the person and an intervention provided to at least one component included in the manufacturing line.

17. The apparatus according to claim 12, wherein the task includes an operation for driving a vehicle, and the intervention includes at least one amongst an intervention directly provided to the person and an intervention provided to at least one component included in the vehicle.

18. The apparatus according to claim 12, wherein the task includes an activity performed by the person, and the intervention includes a healthcare support feedback provided to the person.

19. The apparatus according to claim 12, further including a monitoring unit configured to monitor effectiveness of an intervention on the basis of the determined intervention effect index.

20. The apparatus according to claim 12, wherein the first processor is further configured to:
determine a first performance value in executing the task based on said first sensing information obtained before the intervention is applied,
determine a second performance value in executing the task based on said first sensing information obtained after the intervention is applied,
determine said performance value difference based on the difference between said first performance value and said second performance value.

21. The apparatus according to claim 13, further comprising a processor configure to:
determine an intervention effect index value in correspondence of a person;
select, from the database, an intervention to be applied to a person based on the determined intervention effect index value.

22. The apparatus of claim 13, wherein the database further stores attributes of a person in association with said intervention and said respective intervention effect index, the apparatus is configured to selecting an intervention on the basis of at least one of said stored attributes.

23. A non-transitory computer readable medium computer program including instructions which, when executed on a computer, cause the computer to execute the steps according to claim 1.

24. A signal carrying instructions which, when executed on a computer, cause the computer to execute the steps according to claim 1.

25. A system including an apparatus according to claim 12, and a portable intervention applicator configured for being coupled to a person, wherein the portable intervention applicator is configured to apply to the person an intervention on the basis of information received from the apparatus.
